(19) United States
(12) Patent Application Publication
(10) Pub. No.: US 2005/0238216 A1
(43) Pub. Date: Oct. 27, 2005

(54) Medical image processing apparatus and medical image processing method

(76) Inventor: Sadato Yoden, Kadoma-shi (JP)

Correspondence Address: WENDEROTH, LIND & PONACK, L.L.P., 2033 K STREET N. W., SUITE 800, WASHINGTON, DC, 20006-1021, US

(21) Appl. No.: 11/050,701

(22) Filed: Feb. 7, 2005

(30) Foreign Application Priority Data
Feb. 9, 2004 (JP) 2004-032658

(51) Int. Cl. G06K 9/00; A61B 8/00; A61B 8/12; A61B 8/14

(52) U.S. Cl. 382/128

(57) ABSTRACT

In a medical image processing apparatus 100 that is capable of extracting a contour of an organ or the like in a highly accurate manner within a short time, an image input unit 101 accepts ultrasound images or the like. An electrocardiographic information input unit 102 accepts electrocardiographic information (e.g. ECG waveform). A storage unit 103 stores image data representing the ultrasound images and the electrocardiographic information in association with each other. A dictionary storage unit 104 stores image patterns (dictionary images) used for pattern matching. A pattern dictionary selection unit 105 selects a dictionary image used for pattern matching with reference to the electrocardiographic information. A pattern search range setting unit 106 specifies a range in which pattern matching is to be performed, based on the electrocardiographic information. A cardiac area identification unit 107 detects a cardiac area in the image. A pattern comparison unit 108 identifies the position of each characteristic area in the image by pattern matching. A contour extraction unit 109 extracts a contour based on the position of each characteristic area and the ultrasound image. A display unit 110 displays the extracted contour.

### BACKGROUND OF THE INVENTION

(1) Field of the Invention

The present invention relates to a medical image processing technology, and particularly to an image processing technology for identifying a region of interest, extracting a contour, and the like from medical images of an organ or the like such as a heart whose activity is cyclic, the medical images being obtained on a time series basis.

(2) Description of the Related Art

Conventionally, in the case where a region of interest is identified in and the contour of an organ or the like is extracted from an image of a living body (e.g. ultrasound image and X-ray computed tomogram (CT) image), the spatial position of the obtained ultrasound image is identified first by means of pattern matching or the like. Then, pattern matching and a specification of each characteristic area using a pointing device are performed in combination, in addition to binarization, edge detection, or the like. The above pattern matching is performed by using the evaluation value as a similar reference image out of standard images for research use that are previously created based on an apical four chamber view of a heart being the search target (such reference image(s) are hereinafter referred to as “dictionary image(s)”) (for example, refer to Japanese Laid-Open Patent application No. 2002-140689 (Related art 1) and Japanese Laid-Open Patent application No. 2002-140690 (Related art 2)). In the case of Related art 1, for example, the contour of an organ or the like that is difficult to be identified just by performing edge processing and binarization processing is extracted with reference to the position of a valve and then by further correcting the extracted contour.

However, according to the conventional arts such as Related arts 1 and 2, due to a small number of variations of dictionary images, the same dictionary image is used for most of the ultrasound images in many cases regardless of their positions in the organ or the like. This causes a problem that the accuracy of processing such as the identification of a region of interest becomes unstable, since the similarity can be low between the dictionary image and an ultrasound image being compared. Furthermore, while the above conventional arts are capable of narrowing the search range with reference to the position of a valve, it is possible that a search is not performed appropriately or that it takes a long time for making a search because the same search range is employed for all ultrasound images that have been shot at different timings.

In other words, since the above conventional arts use the same dictionary image regardless of the positions of the respective ultrasound images in an organ or the timing at which such ultrasound images have been shot. This causes a problem that the accuracy of contour extraction becomes low because a desirable result cannot be achieved for searches in ultrasound images corresponding to different positions and cycles. Furthermore, since a fixed search range is applied for all frames, it takes long for performing a search if a wide search range is set.

### SUMMARY OF THE INVENTION

The present invention has been conceived in view of the above problems, and it is an object of the present invention to provide a medical image processing apparatus and a medical image processing method that are capable of extracting a contour of an organ or the like in a highly accurate manner within a short time.

In order to achieve the above object, the medical image processing apparatus according to the present invention is a medical image processing apparatus that extracts a contour of a predetermined part of a subject from a medical image, said apparatus comprising: an image generation unit that generates images in which the predetermined part is shown; a reference image holding unit that holds reference images corresponding to the generated images, the reference images being added with attribute information of the subject; an electrocardiographic information obtainment unit that obtains electrocardiographic information that represents changes in cardiac muscle movement of the subject; an image identification unit that identifies one of the generated images based on the electrocardiographic information; a pattern comparison unit that identifies a position of a characteristic area by comparing the identified image and the reference images; and a contour extraction unit that extracts the contour of the predetermined part from the identified image, based on the identified position of the characteristic area.

Since the present invention makes it possible to perform pattern matching only for a limited image that is identified based on electrocardiographic information and therefore to perform a pattern comparison in an accurate manner within a short time, it eventually becomes possible to extract a contour of an organ or the like in a highly accurate manner at high speed.

Furthermore, in order to achieve the above object, in the above medical image processing apparatus according to the present invention, said image identification unit identifies one of the generated images based on the electrocardiographic information corresponding to one of end-systolic phase and end-diastolic phase.

Since the present invention makes it possible to perform pattern matching only for a further limited image that is identified based on electrocardiographic information and therefore to perform a pattern comparison in an accurate manner within a short time, it eventually becomes possible to extract a contour of an organ or the like in a highly accurate manner at high speed.

It should be noted that the present invention is capable of being embodied as a medical image processing method that includes, as its steps, characteristic constituent elements that make up the above medical image processing apparatus, as well as being capable of causing a personal computer or the like to execute a program that includes all of such steps.

As described above, since the present invention is capable of extracting a contour of an organ or the like of a subject in an accurate manner at high speed in medical diagnosis and measurement, the effects produced by the present invention are enormous in the field of medicine.

The disclosure of Japanese Patent Application No. 2004-032658 filed on Feb. 9, 2004 including specification, drawings and claims is incorporated herein by reference in its entirety.

### BRIEF DESCRIPTION OF THE DRAWINGS

These and other objects, advantages and features of the invention will become apparent from the following description thereof taken in conjunction with the accompanying drawings that illustrate a specific embodiment of the invention. In the Drawings:

FIG. 1 is a block diagram showing a functional structure of a medical image processing apparatus according to a first embodiment;

FIG. 2 is a flowchart showing a flow of processes performed by the medical image processing apparatus according to the first embodiment;

FIG. 3 is a diagram showing an example of a typical electrocardiogram (ECG);

FIG. 4 is a diagram showing how a pattern dictionary selection unit selects an appropriate dictionary image according to a waveform of an ECG received from a dictionary storage unit;

FIG. 5 is a diagram showing the positions of characteristic areas;

FIGS. 6A and 6B are diagrams showing search ranges for the respective characteristic areas according to a conventional art that uses no ECG;

FIGS. 7A and 7B are diagrams showing search ranges for the respective characteristic areas that are narrowed by use of an ECG;

FIG. 8 is a block diagram showing a functional structure of a medical image processing apparatus according to a second embodiment;

FIG. 9 is a flowchart showing a flow of processes performed by the medical image processing apparatus according to the second embodiment;

FIGS. 10A and 10B are diagrams showing an example of positions specified by an operator;

FIG. 11 is a block diagram showing a functional structure of a medical image processing apparatus according to a third embodiment; and

FIG. 12 is a flowchart showing a flow of processes performed by the medical image processing apparatus according to the third embodiment.

### DESCRIPTION OF THE PREFERRED EMBODIMENTS

The following describes the embodiments of the present invention with reference to the drawings.

### First Embodiment

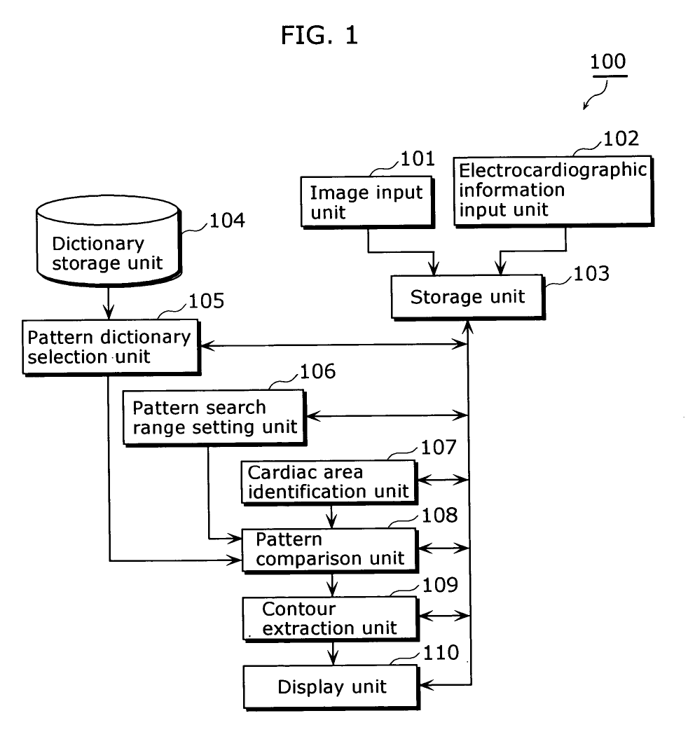

FIG. 1 is a block diagram showing a functional structure of a medical image processing apparatus 100 according to the first embodiment. The medical image processing apparatus 100 is a diagnostic apparatus for medical use, such as an ultrasonic diagnostic apparatus that generates ultrasound images based on echo signals of ultrasonic pulses emitted to a subject, an X-ray CT apparatus that generates tomograms based on the amount of X rays passing through a subject, and an MRI apparatus that generates magnetic resonance (MR) images based on electromagnetic waves released from a subject. The medical image processing apparatus 100 extracts an internal contour of an organ (e.g. heart and blood vessel) whose activity is cyclic, using moving images of such organ and an electrocardiogram (ECG) obtained from the subject, and displays the extracted contour or the like. Note that, for convenience sake, the following descriptions are provided on the assumption that the medical image processing apparatus 100 is an ultrasonic diagnostic apparatus that generates ultrasound images of a heart at the speed of 30 frames per second.

As shown in FIG. 1, the medical image processing apparatus 100 is comprised of an image input unit 101, an electrocardiographic information input unit 102, a storage unit 103, a dictionary storage unit 104, a pattern dictionary selection unit 105, a pattern search range setting unit 106, a cardiac area identification unit 107, a pattern comparison unit 108, a contour extraction unit 109, and a display unit 110.

The image input unit 101 accepts ultrasound images that are generated based on, for example, echo signals received via an ultrasound probe (not illustrated in the drawing). The electrocardiographic information input unit 102 obtains, from the subject, electrocardiographic information (e.g. data representing an ECG waveform in the time domain) via an electrode or the like that is put on the subject's hand, feet, or chest. Furthermore, the electrocardiographic information input unit 102 accepts, from the operator, attribute information related to the subject (e.g. age, height, weight, sex, body type, symptom, and the name of a disease).

The storage unit 103 is equipped with a hard disk device (HDD), for example, and stores the following information in association with one another: image data representing the ultrasound images inputted to the image input unit 101; the electrocardiographic information obtained via the electrocardiographic information input unit 102; and the attribute information of the subject. The dictionary storage unit 104, which is a random access memory (RAM), an HDD, or the like, holds dictionary data for dictionary images used for pattern matching.

The pattern dictionary selection unit 105 selects a dictionary image corresponding to the timing specified by the operator (or a predetermined timing) for use for pattern matching, with reference to the electrocardiographic information stored in the storage unit 103. The pattern search range setting unit 106 identifies (or limits) a range, in the input ultrasound image, within which pattern matching is to be performed. In this case, the range that is slightly larger than the size of the dictionary image is to be identified. The cardiac area identification unit 107 accepts, from the operator using a mouse or the like, a specification of the area in the inputted ultrasound image that includes the Left ventricle. The pattern comparison unit 108 detects the position of each characteristic area of the heart in the ultrasound image by means of pattern matching that utilizes dictionary image. The contour extraction unit 109 extracts, from the ultrasound image, the contour (e.g. an inner contour) of the ventricle or the like, based on the detected positions of the respective characteristic areas. In so doing, the contour extraction unit 109 may extract an initial contour first with reference to the detected positions of the respective characteristic areas, and then extract a more precise contour. The display unit 110, which is a cathode-ray tube (CRT), for example, displays the ultrasound image or the like in which the extracted contour is included.

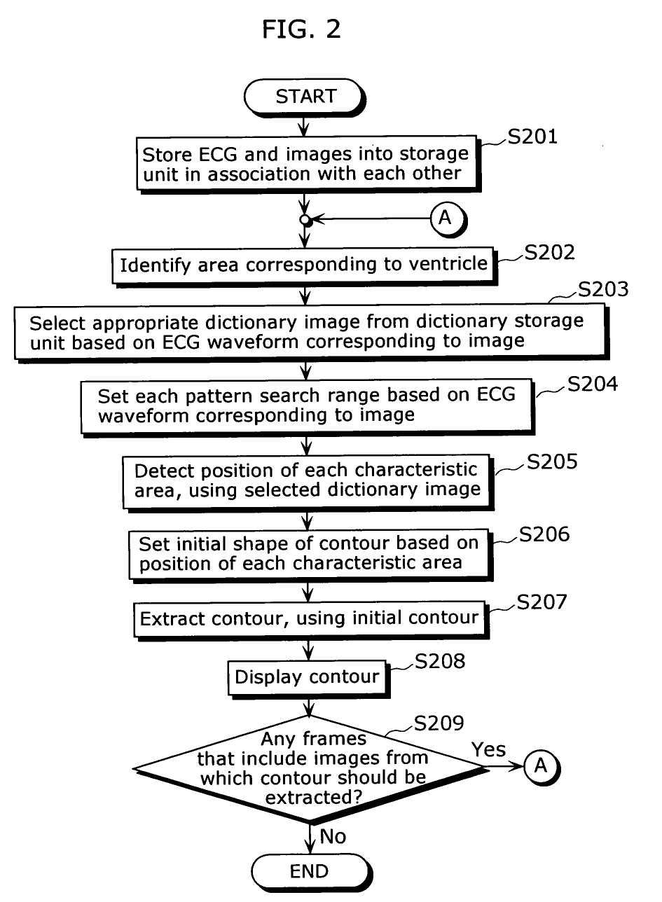

Next, a description is given of operations of the medical image processing apparatus 100 with the above structure. FIG. 2 is a flowchart showing a flow of processes performed by the medical image processing apparatus 100.

First, the storage unit 103 stores, in association with each other, image data representing moving images (ultrasound images) obtained by the image input unit 101 and electrocardiographic information that have been obtained by the electrocardiographic information input unit 102 together with such image data (S201). Next, the cardiac area identification unit 107 identifies an ultrasound image of the heart that corresponds to a range and timing specified by the operator (such ultrasound image is hereinafter referred to as “frame image”) (S202).

Next, after the cardiac area identification unit 107 identifies the frame image to be processed, the pattern dictionary selection unit 105 selects a dictionary image that is appropriate for identifying the position of each characteristic area (S203). More specifically, the pattern dictionary selection unit 105 judges how the heart is contracting from the electrocardiographic information corresponding to the identified frame image stored in the storage unit 103, and selects an appropriate dictionary image from among those stored in the dictionary storage unit 104. The dictionary storage unit 104 holds various dictionary images, on a per-characteristic area basis, corresponding to cardiac cycles starting from the end-systolic phase to the end-diastolic phase.

Here, a description is given of an ECG used in the present embodiment.

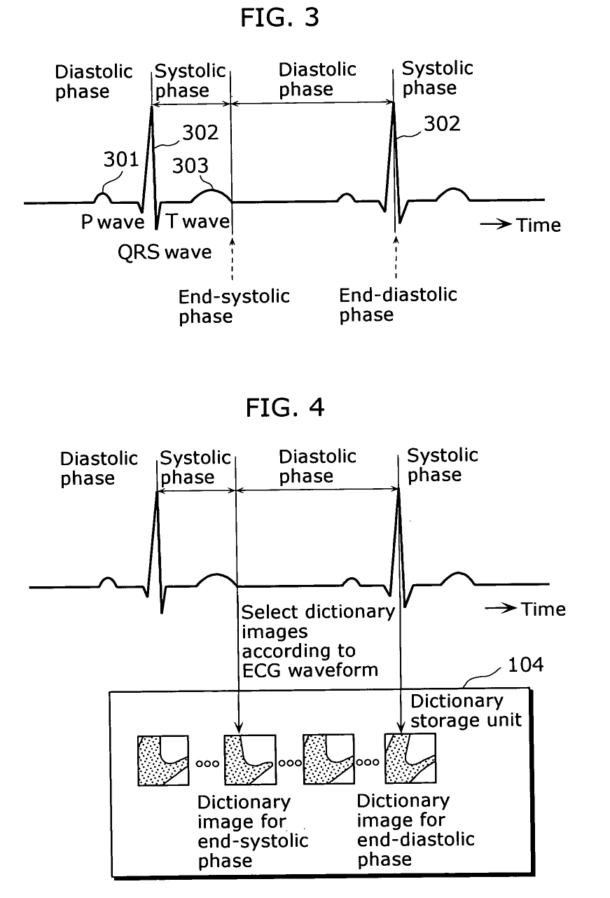

FIG. 3 is a diagram showing a waveform in a typical ECG. In general, waveform signals known as a P wave 301, a QRS wave 302, and a T wave 303 are observed in an ECG. The occurrence of the QRS wave 302 marks the beginning of cardiac muscle contraction. A period from the beginning to the end of cardiac muscle contraction is referred to as “systole”. When the contraction is complete, the T wave 303 is measured. The timing at which the T wave ends is referred to as the “end-systolic phase”. After the end-systolic phase, the cardiac muscle becomes relaxed, and it enters diastole in which cardiac volume increases. From then on, the cardiac muscle contraction continues until it begins to dilate at the timing indicated by the occurrence of the QRS wave 302. The timing indicated by the peak of QRS wave 302 is referred to as the “end-diastolic phase”. The pattern dictionary selection unit 105 selects appropriate dictionary images based on the respective timings corresponding to the end-systolic phase and the end-diastolic phase.

FIG. 4 is a diagram showing how the pattern dictionary selection unit 105 selects an appropriate dictionary image according to a waveform of an ECG received from the dictionary storage unit 104. By selecting and using an appropriate dictionary image, it becomes possible to detect each characteristic area in a more accurate manner, compared with a conventional method that uses the common dictionary image.

Note that a higher accuracy can be achieved by generating dictionary images and selecting an appropriate dictionary image from such generated dictionary images that are categorized, on an item basis (for each of at least one item), according to the subject's age, height weight, sex, body type (e.g. thin build, standard type, corpulent), symptom (e.g. cardiac angina, valvular heart disease, cardiomyopathy), and the name of a disease, as well as being categorized according to timings indicated by a plurality of characteristic waveform signals in an ECG.

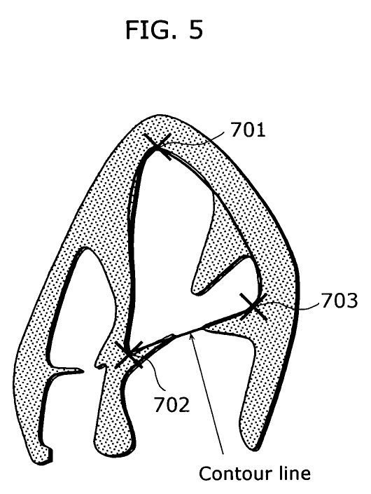

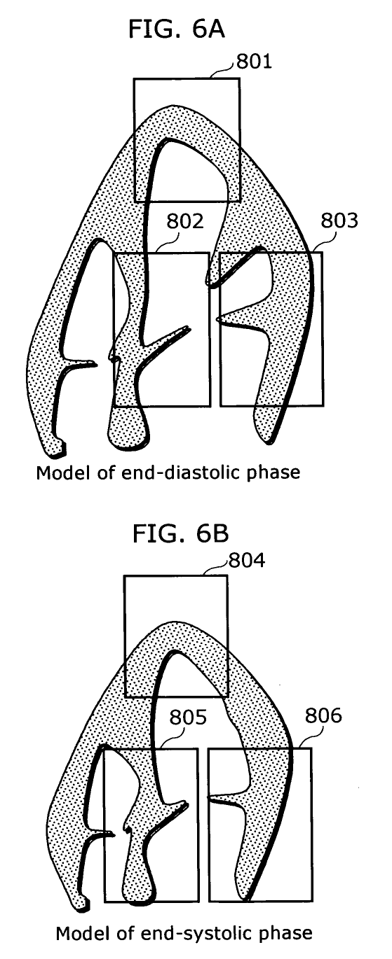

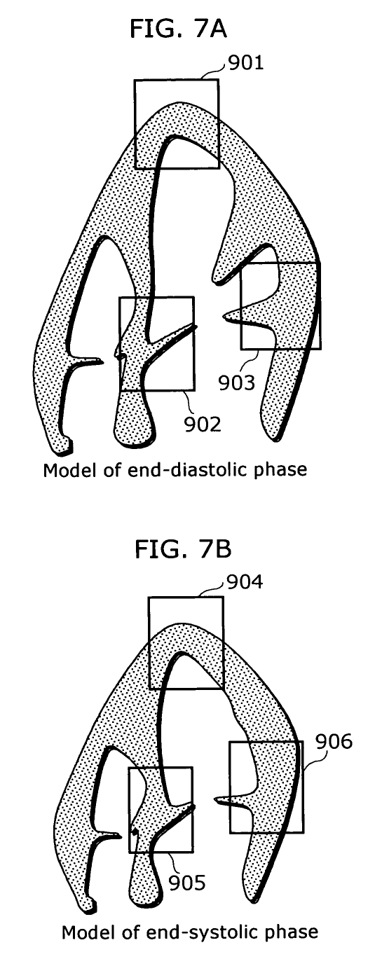

Then, the pattern search range setting unit 106 judges how the heart is contracting based on the electrocardiographic information corresponding to the identified frame image, and sets each search range in which pattern matching is to be performed (S204). The movement of the heart is cyclic, and so is the movement of each characteristic area. In the case of characteristic areas shown in FIG. 5, for example, a conventional method sets a search range for each characteristic area as illustrated in FIGS. 6A and 6B, but the present embodiment is capable of further narrowing a search range for each characteristic area by selecting a dictionary image that corresponds to the timing indicated by each characteristic waveform signal in an ECG and thus capable of having search ranges as illustrated in FIGS. 7A and 7B. Accordingly, by identifying an ultrasound image according to a waveform of an ECG, it becomes possible to shorten the time required for search since it is possible to narrow a range in the ultrasound image for which pattern matching is to be performed.

Furthermore, the pattern comparison unit 108 performs pattern matching based on the above-selected dictionary image and the above-set search range so as to identify each characteristic area (S205). Here, after the pattern dictionary selection unit 105 selects dictionary images from those stored in the dictionary storage unit 104, the pattern comparison unit 108 identifies the positions of the respective characteristic areas by performing pattern matching within the respective ranges set by the pattern search range setting unit 106, by use of the identified ultrasound images stored in the storage unit 103 and the selected dictionary images. Note that this pattern matching can be any of template matching, subspace method, and complex similarity method.

Next, the contour extraction unit 109 extracts an initial contour of the inner shape of the ventricle based on the characteristic areas that have been identified as being included in the above-set cardiac area (S206). Furthermore, the contour extraction unit 109 extracts an inner contour of the Left ventricle, based on the extracted initial contour (S207). Here, the contour line is extracted by detecting an edge, in the frame image, near the initial contour. The display unit 110 displays the detected contour together with the frame image stored in the storage auntie 103, and presents them to the operator (S208). When it is necessary to extract a contour in another frame image, the above processes (S202 to S208) are continuously performed.

As described above, the present embodiment is capable of shortening the time required for pattern matching since it is possible to narrow a range in an ultrasound image for which pattern matching should be performed, by selecting dictionary images that correspond to timings indicated by the respective characteristic waveform signal in an ECG.

### Second Embodiment

The first embodiment describes an embodiment in which a pattern comparison is performed using a previously prepared dictionary image. The second embodiment describes an embodiment in which pattern comparison is performed using an ultrasound image or the like that is obtained in real-time at examination time.

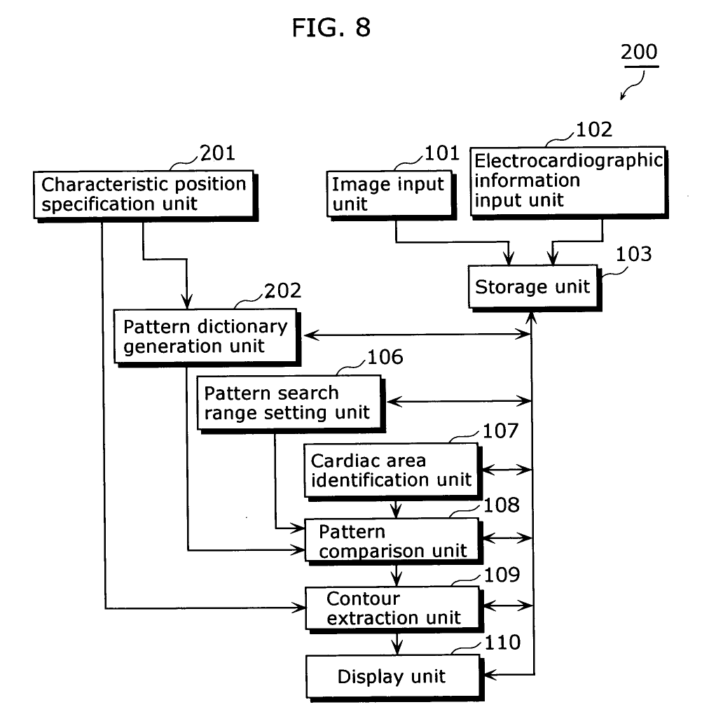

FIG. 8 is a block diagram showing a functional structure of a medical image processing apparatus 200 according to the second embodiment. The medical image processing apparatus 200, an example of which is an ultrasonic diagnostic apparatus as in the case of the medical image processing apparatus 100 of the first embodiment, is an imaging diagnostic apparatus that extracts an inner contour of a heart or the like using an ECG and moving images of the heart generated at the speed of 30 frames per second, and displays the extracted contour or the like. Note that the components that are the same as those described in the first embodiment are assigned the same numbers and descriptions thereof are omitted.

As shown in FIG. 8, the medical image processing apparatus 200 is comprised of an image input unit 101, an electrocardiographic information input unit 102, a storage unit 103, a characteristic position specification unit 201, a pattern dictionary generation unit 202, a pattern search range setting unit 106, a cardiac area identification unit 107, a pattern comparison unit 108, a contour extraction unit 109, and a display unit 110.

The characteristic position specification unit 201 accepts an operator's specification of the position of each characteristic area. The pattern dictionary generation unit 202 generates a dictionary image for the position of each characteristic area specified by the operator.

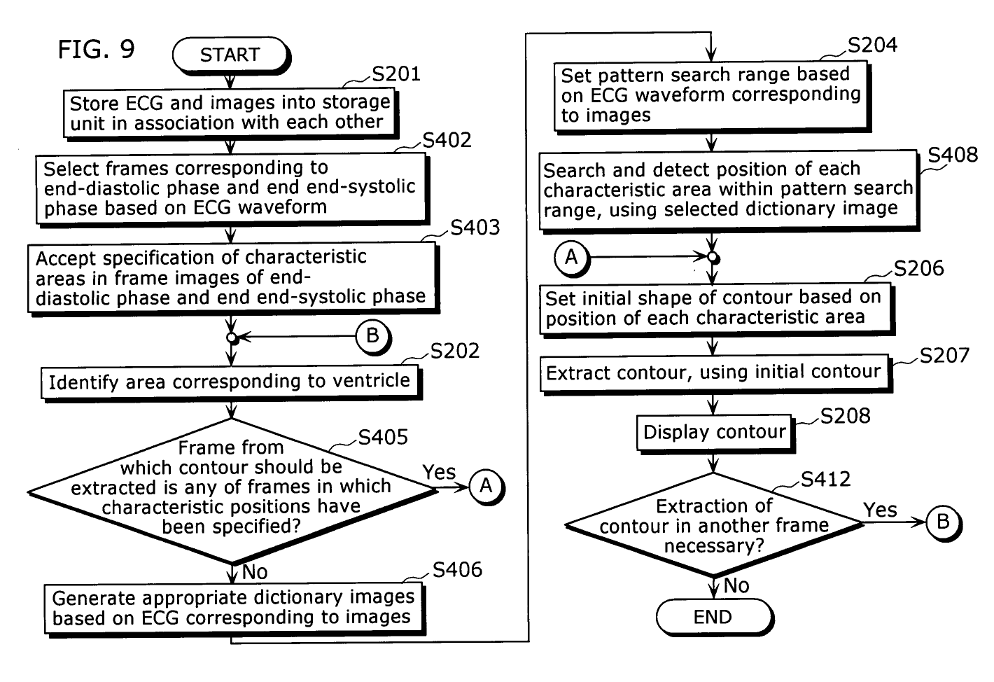

Next, a description is given of operations of the medical image processing apparatus 200 with the above structure. FIG. 9 is a flowchart showing a flow of processes performed by the medical image processing apparatus 200.

First, moving images inputted via the image input unit 101 and electrocardiographic information inputted from the electrocardiographic information input unit 102 together with the moving images are stored into the storage unit 103 in association with each other (S201). Then, based on the electrocardiographic information, the display unit 101 selects frame images corresponding to the end-diastolic and end-systolic phases from the storage unit 103, and displays the selected frame images (S402).

Next, when accepting, via the characteristic position specification unit 201, a specification of each characteristic area from the operator looking at the display unit 101 (S403), the cardiac area identification unit 107 identifies an area corresponding to a ventricle from which a contour is to be extracted (S202). In response to this, the pattern dictionary generation unit 202 judges whether or not the frame images in which the positions of the characteristic areas have been specified by the characteristic position specification unit 201 include the frame image from which a contour should be extracted (S405).

First, a description is given for the case where the judgment is made in S405 that the frame is not the one in which the position of each characteristic area is specified.

In order to extract the inner shape of the ventricle, the contour extraction unit 109 first extracts an initial contour, making a correction to the frame image on the basis of the cardiac area set in S202 and the position of each characteristic area specified in S403 (S206). Then, the contour extraction unit 109 extracts an inner contour of the ventricle using the above-generated initial contour (S207). Here, a contour line is extracted by detecting an edge, in the frame image, near the initial contour. The display unit 110 displays the frame image together with the extracted contour (S208). When it is necessary to extract a contour in another frame image, the above processes (S202 to S412) are continuously performed.

Next, a description is given of the case where the judgment is made in S405 that the frame image is not the one in which the position of each characteristic area is specified.

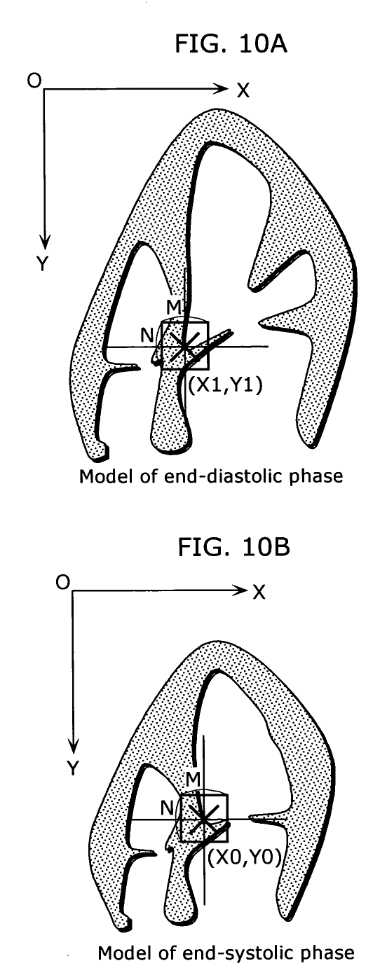

When a cardiac area is specified, the pattern dictionary generation unit 202 generates dictionary images that are appropriate for detecting the positions of the respective characteristic areas (S406). For example, referring to FIGS. 10A and 10B, suppose that the operator has specified, in the frame images corresponding to the end-diastolic phase and the end-systolic phase, a position P (X0, Y0) and a position Q (X1, Y1). In this case, image patterns with the size of M×N (pixels) are extracted to be used as basic patterns, by centering on the position P (X0, Y0) and the position Q (X1, Y1) which have been specified as the positions of the respective characteristic areas in the above two frame images. Then, a calculation is performed, based on the electrocardiographic information stored in the storage unit 103, to determine the distance between the frame image from which a contour is to be extracted and the two frame images corresponding to the end-diastolic phase and the end-systolic phase in which the characteristic areas have been specified. Then, a dictionary image used for search is generated based on the generated two basic patterns and on the calculated distance from the frame images corresponding to the end-diastolic phase and the end-systolic phase. Letting the positions of the frame images corresponding to the end-systolic phase and end-diastolic phase in which the positions of the respective characteristic areas have been specified be “0” and “1”, respectively, and the position of a dictionary image to be generated be “t”, their respective patterns are represented as P₀, P₁, and Pₜ. In the case where alpha blending is used (“t” is regarded as “α”), pattern Pₜ can be represented by the following equation (1), where 0≦x<M, 0≦y<N, and 0≦t≦1:
Pₜ(x, y)=(1−t)P₀(x, y)+tP₁(x, y) (1)

Note that other than alpha blending, morphing or the like may be used for the generation of a dictionary image used for search. Then, the pattern search range setting unit 106 judges how the heart is contracting, based on the electrocardiographic information stored in the storage unit 103, and sets each search range in which pattern matching is to be performed (S204). The search range is set based on the positions specified as the positions of characteristic areas corresponding to the end-diastolic phase and end-systolic phase, in consideration of the timings indicated by waveform signals in the ECG. Then, the characteristic areas are detected from the search range that has been identified using the generated dictionary image (S408). The detection of the characteristic areas are performed by making a comparison between (i) the dictionary image that has been generated based on images and electrocardiographic information stored in the storage unit 103 as well as on a specification of each characteristic area accepted by the characteristic position specification unit 201 and (ii) images stored in the storage unit 103. Note that this comparison may be performed using any of template matching, subspace method, and complex similarity method, as in the case of the first embodiment. Accordingly, it becomes possible to detect the position of each characteristic area in the frame image. The subsequent processes are the same as those described above (S206 to S412).

As described above, the present embodiment is capable of extracting a contour in a more accurate manner since it newly generates a dictionary image to use it for pattern matching in the case where there is no appropriate dictionary image corresponding to the timing indicated by a characteristic waveform in an ECG at the time of contour extraction.

### Third Embodiment

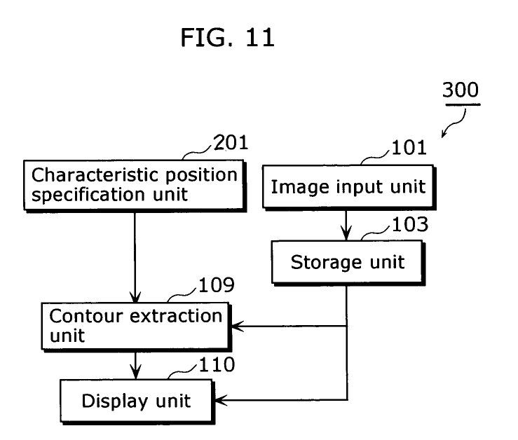

FIG. 11 is a block diagram showing a functional structure of a medical image processing apparatus 300 according to the third embodiment. The third embodiment describes an example usage of a contour extraction method, and the present medical image processing apparatus 300 is an imaging diagnostic apparatus that accepts ultrasound images of a heart or the like and extracts and displays an inner contour of the heart.

Referring to FIG. 11, the input unit 101 accepts image data. The storage unit 103 holds the image data. The characteristic position specification unit 201 accepts an operator's specification of the position of each characteristic area. The contour extraction unit 109 extracts an inner contour of a ventricle based on the specified characteristic positions and images stored in the storage unit 103. The display unit 110 displays the extracted contour, the image information and the like.

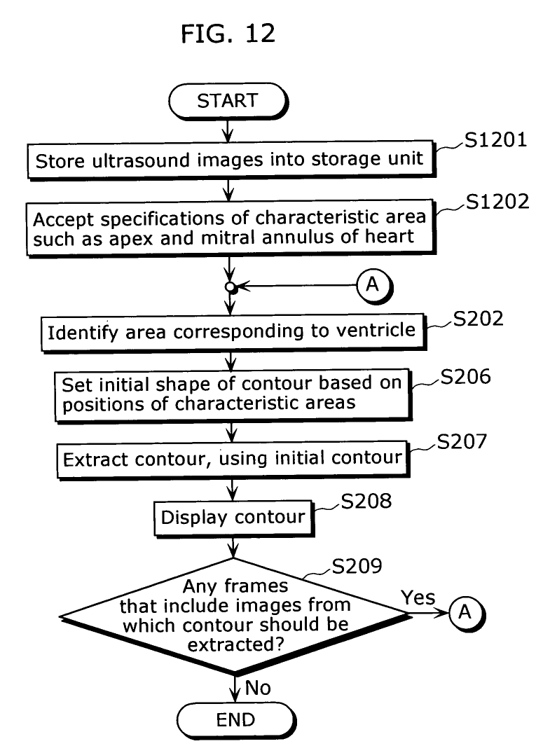

Next, a description is given of operations of the medical image processing apparatus 300 with the above structure. FIG. 12 is a flowchart showing a flow of processes performed by the medical image processing apparatus 300.

First, ultrasound images of the heart are inputted to the image input unit 101 to be stored into the storage unit 103 (S1201). Next, an operator's specification of the position of each characteristic area (e.g. apex and mitral annulus) is accepted via the characteristic position specification unit 201 (S1202) so as to identify an area of a ventricle to be examined, based on the specified positions of the respective characteristic areas (S202).

An initial contour, which is used to extract an inner shape of the ventricle, is set after being corrected based on the area of the ventricle identified in S202 and on the positions of the respective characteristic areas specified in S1202 (S206). Next, the contour extraction unit 109 extracts an inner contour of the ventricle, using the initial contour that has been corrected on the basis of the positions of the respective characteristic areas (S207). Here, a contour line is extracted by detecting an edge, in the frame image, near the initial contour. The display unit 110 displays the ultrasound image including the contour, based on the contour extracted in S207 and the identified image stored in the storage unit 103 (S208). When it is necessary to extract a contour in another frame image, the above processes (S202 to S209) are continuously performed.

Note that although the first, second, and third embodiments describe the case where an image input apparatus for obtaining an image (e.g. imaging apparatus) is implemented separately from the ultrasonic diagnostic apparatus 100/200/300, but the present invention is applicable to the case where the image input apparatus is integrated into the ultrasonic diagnostic apparatus 100/200/300. Furthermore, the processes according to the present invention may be implemented as software by using a personal computer or the like having image input function.

Although only some exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention.

### INDUSTRIAL APPLICABILITY

The present invention is applicable to an ultrasonic diagnostic apparatus that handles an ECG, and other apparatuses such as an X-ray CT apparatus and an MRI apparatus capable of processing images that are synchronous with ECG waveform cycles.

1. A medical image processing apparatus that extracts a contour of a predetermined part of a subject from a medical image, said apparatus comprising:
an image generation unit operable to generate images in which the predetermined part is shown;
a reference image holding unit operable to hold reference images corresponding to the generated images, the reference images being added with attribute information of the subject;
an electrocardiographic information obtainment unit operable to obtain electrocardiographic information that represents changes in cardiac muscle movement of the subject;
an image identification unit operable to identify one of the generated images based on the electrocardiographic information;
a pattern comparison unit operable to identify a position of a characteristic area by comparing the identified image and the reference images; and
a contour extraction unit operable to extract the contour of the predetermined part from the identified image, based on the identified position of the characteristic area.

2. The medical image processing apparatus according to claim 1,
wherein said image identification unit is operable to identify one of the generated images based on the electrocardiographic information corresponding to one of end-systolic phase and end-diastolic phase.

3. The medical image processing apparatus according to claim 1, further comprising, in replacement of said pattern comparison unit,
a characteristic position accepting unit operable to accept a specification of the position of the characteristic area included in the identified image,
wherein said contour extraction unit is operable to extract the contour of the predetermined part from the identified image, based on the specified position of the characteristic area.

4. The medical image processing apparatus according to claim 1,
wherein said reference image holding unit is further operable to generate a reference image based on the images generated by said image generation unit, and to hold the generated reference image.

5. The medical image processing apparatus according to claim 1,
wherein the attribute information of the subject includes at least one of the following attributes: age, height, weight, sex, body type, symptom, and a name of a disease, and
the medical image processing apparatus further comprises
a reference image selection unit operable to accept a specification of at least one of the attributes, and to select one of the reference images based on the accepted specification,
wherein said pattern comparison unit is operable to perform the comparison using the selected reference image.

6. The medical image processing apparatus according to claim 1,
wherein said image generation unit is operable to generate the images based on an echo signal of an ultrasonic pulse transmitted to the subject.

7. The medical image processing apparatus according to claim 1,
wherein said image generation unit is operable to generate the images based on an amount of X-rays passing through the subject.

8. The medical image processing apparatus according to claim 1,
wherein said image generation unit is operable to generate the images based on an electromagnetic wave released from the subject.

9. A medical image processing method for extracting a contour of a predetermined part of a subject from a medical image, said method comprising:
generating images in which the predetermined part is shown;
obtaining electrocardiographic information that represents changes in cardiac muscle movement of the subject;
identifying one of the generated images based on the electrocardiographic information;
identifying a position of a characteristic area by comparing the identified image and reference images corresponding to the generated images, the reference images being added with attribute information of the subject; and
extracting the contour of the predetermined part from the identified image, based on the identified position of the characteristic area.

10. The medical image processing method according to claim 9, further comprising, in replacement of said identifying of the position of the characteristic area,
accepting a specification of the position of the characteristic area included in the identified image,
wherein in said extracting of the contour, the contour of the predetermined part is extracted from the identified image, based on the specified position of the characteristic area.

11. The medical image processing method according to claim 9,
wherein the characteristic area is Apex.

12. The medical image processing method according to claim 9,
wherein the attribute information of the subject includes at least one of the following attributes: age, height, weight, sex, body type, symptom, and a name of a disease, and
the medical image processing method further comprises
accepting a specification of at least one of the attributes, and selecting one of the reference images based on the accepted specification,
wherein in said identifying of the position of the characteristic area, the comparison is performed using the selected reference image.

13. A program used for a medical image processing method for extracting a contour of a predetermined part of a subject from a medical image, said program causing a computer to execute:
generating images in which the predetermined part is shown;
obtaining electrocardiographic information that represents changes in cardiac muscle movement of the subject;
identifying one of the generated images based on the electrocardiographic information;
identifying a position of a characteristic area by comparing the identified image and reference images corresponding to the generated images, the reference images being added with attribute information of the subject; and
extracting the contour of the predetermined part from the identified image, based on the identified position of the characteristic area.

14. The program according to claim 13, further causing the computer to execute, in replacement of said identifying of the position of the characteristic area,
accepting a specification of the position of the characteristic area included in the identified image,
wherein in said extracting of the contour, the contour of the predetermined part is extracted from the identified image, based on the specified position of the characteristic area.
